(19) United States
(12) Patent Application Publication
(10) Pub. No.: US 2023/0358735 A1
(43) Pub. Date: Nov. 9, 2023

(54) VIRAL DETECTION SYSTEM AND METHODS OF USING SAME

(71) Applicant: PURDUE RESEARCH FOUNDATION, West Lafayette, IN (US)

(72) Inventors: Lia Stanciu-Gregory, West Lafayette, IN (US); Winston Yen-Yu Chen, Los Altos, CA (US); Amit Kumar Barui, West Lafayette, IN (US); Ana Maria Ulloa Gomez, Lafayette, IN (US)

(73) Assignee: PURDUE RESEARCH FOUNDATION, West Lafayette, IN (US)

(21) Appl. No.: 18/048,597

(22) Filed: Oct. 21, 2022

Related U.S. Application Data
(60) Provisional application No. 63/270,962, filed on Oct. 22, 2021.
(60) Provisional application No. 63/270,943, filed on Oct. 22, 2021.

(51) Int. Cl. G01N 33/543; B82Y 15/00; G01N 33/569

(52) U.S. Cl. CPC G01N 33/5438; B82Y 15/00; G01N 33/56983; G01N 2800/26

(57) ABSTRACT

A biosensing device and methods for detecting the SARS-CoV-2 virus sequence in a biological fluid. The device includes an interdigitated electrode, a supporting flexible membrane and an electrode material that is disposed on the interdigitated electrode and supporting flexible membrane. The electrode material is selected from (i) two-dimensional and layered MXenes and (2) printable graphene, wherein the electrode material is functionalized with ssDNA primers specific to the SARS-CoV-2 virus sequence to be detected. The device also includes a sensor that reads an electrical resistance change across the interdigitated electrode after a target DNA sequence is applied to the electrode material.

### CROSS REFERENCE TO RELATED APPLICATIONS

This application claims priority to U.S. Provisional Application No. 63/270,943 filed on Oct. 22, 2021, and U.S. Provisional Application No. 63/270,962 filed on Oct. 22, 2021, both are incorporated herein by reference in their entirety.

### SEQUENCE LISTING XML

This application includes a Sequence Listing XLM, named 69260-02_PRF-009_Sequence_Listing_XML_06152023, created on Jun. 15, 2023, with a file size of 3,255 bytes, and is incorporated by reference in its entirety.

### BACKGROUND

### Field

Embodiments provided herein relate to biosensing. More particularly, embodiments provided herein relate to biosensors for SARS-CoV-2 viral RNA and uses thereof.

### Description of the Related Art

Coronavirus disease 2019 (COVID-19) is an emerging human infectious disease caused by severe acute respiratory syndrome 2 (SARS-CoV-2, initially called novel coronavirus 2019-nCoV) virus. A global emergency outbreak of COVID-19 challenged both health care personnel and the medical facilities worldwide with more than 200 countries affected by the pandemic. Thus, an accurate and specific diagnosis of COVID-19 is urgently needed for effective point-of-care detection and disease management.

Coronavirus disease 2019 (COVID-19) pandemic was first detected in December 2019 from Wuhan City of China and has caused a global outbreak and a serious public health issue. The novel coronavirus was named severe acute respiratory syndrome 2 (SARS-CoV-2, initially named 2019-nCoV), the pathogen causing COVID-19, which causes respiratory and intestinal illness in both humans and animals. As of 1st October 2020, the rapid spread of SARS-CoV-2, has impacted more than 200 countries, infecting more than thirty-million people with over one-million confirmed deaths. COVID-19/SARS-CoV-2 is still devastating the people and countries around the world and seems to be difficult to tackle at this moment. There have been recurrent outbreaks from emerging coronavirus such as Severe Acute Respiratory Syndrome (SARS-CoV) in 2003 and Middle East Respiratory Syndrome (MERS-CoV) in 2012. Thus, there is a critical need for the development of a rapid, inexpensive, and reliable identification method toward novel viruses that can greatly facilitate public health response to emerging viral threats.

Real-time reverse transcription polymerase chain reaction (RT-PCR) is one of the most widely used laboratory methods for the detection of SARS-CoV-2 using samples from respiratory secretions. The gene targets for RT-PCR molecular assays developed by various countries are genetically similar. The national RT-PCR protocols among various countries typically target the nucleocapsid (N) gene of SARS-CoV-2 due to their highly conserved nature and less nucleotide changes over time. RT-PCR is highly sensitive and specific method for detecting viral RNA by the amplification of specific regions of sequences. However, molecular diagnosis using RT-PCR method presents some drawbacks, including long processing time, tedious sample preparation, laboratory-based testing, and manpower issues, limiting the capacity of testing all the suspected cases during large-scale outbreaks. Thus, it is urgent to develop a rapid and accurate diagnostic technology for the detection of SARS-CoV-2.

### SUMMARY

A biosensing device for detecting the SARS-CoV-2 virus sequence in a biological fluid, comprising an interdigitated electrode; a supporting flexible membrane; an electrode material that is disposed on the interdigitated electrode and supporting flexible membrane, wherein the electrode material is selected from (1) two-dimensional and layered MXenes and (2) printable graphene, wherein the electrode material is functionalized with ssDNA primers specific to the SARS-CoV-2 virus sequence to be detected; and a sensor that reads an electrical resistance change across the interdigitated electrode after a target DNA sequence is applied to the electrode material.

A method for detecting the SARS-CoV-2 virus sequence in a biological fluid, comprising disposing a biological fluid sample within a sensing device, comprising an interdigitated electrode; a supporting flexible membrane; an electrode material that is disposed on the interdigitated electrode and supporting flexible membrane, wherein the electrode material is selected from (1) two-dimensional and layered MXenes and (2) printable graphene, wherein the electrode material is functionalized with ssDNA primers specific to the SARS-CoV-2 virus sequence to be detected; and a sensor that reads an electrical resistance change across the interdigitated electrode after a target DNA sequence is applied to the electrode material; waiting for a residence time; and reading an electrical signal from the sensor.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are, therefore, not to be considered limiting of its scope, for the invention may admit to other equally effective embodiments. It is emphasized that the figures are not necessarily to scale and certain features and certain views of the figures can be shown exaggerated in scale or in schematic for clarity and/or conciseness.

FIG. 1 depicts different generations (“Gen”) of sensors tested in Example 1: Gen 1, Gen 2, Gen 3, Gen 4, Gen 5.

FIG. 2 depicts a working principle of the developed sensor.

FIG. 3A depicts a SARS-CoV-2 electrochemical sensor being functionalized with DNA ligand probe.

FIG. 3B depicts a sensor response in model buffer for negative control and SARS-CoV-2 spiked samples.

FIG. 3C depicts a sensor response in model clinical matrix, artificial saliva for detection of severe acute respiratory syndrome-related coronavirus 2 (SARS-CoV-2) strain 2019-n-CoV/USA-WA1/2020 spiked (10⁵ copies/µL) samples.

FIG. 4 depicts a sensor response of a quadruplex SARS-CoV-2 sensor array for negative and positive control samples.

FIG. 5A depicts cross reactivity of the developed assay using artificial saliva.

FIG. 5B depicts cross reactivity of the developed assay using SARS-CoV spiked (10⁵ copies/µL) artificial saliva.

FIG. 5C depicts cross reactivity of the developed assay using MERS-CoV spiked (10⁵ copies/µL) artificial saliva.

FIG. 5D depicts cross reactivity of the developed assay using severe acute respiratory syndrome-related coronavirus 2 (SARS-CoV-2) strain 2019-n-CoV/USA-WA1/2020 spiked (10⁵ copies/µL) artificial saliva.

FIG. 6 depicts a normalized sensor response upon application of different potentials (0.1-1.1 V).

FIG. 7A depicts a map of the DNA plasmid used as template in the PCR reaction.

FIG. 7B depicts an amplified 72 bp PCR product.

FIG. 8A depicts an ssDNA primer concentration optimization with a 1 µM concentration of primer used for electrode functionalization.

FIG. 8B depicts an ssDNA primer concentration optimization with a 2 µM concentration of primer used for electrode functionalization.

FIG. 8C depicts an ssDNA primer concentration optimization with a 3 µM concentration of primer used for electrode functionalization.

FIG. 9 depicts printing of graphene with a 1st and 3rd layer in parallel to the IDE fingers and a 2nd layer perpendicular to the IDE fingers.

FIG. 10 depicts a comparison between an EIS of Graphene electrodes with 1st and 3rd layer of graphene printed in parallel to the IDE fingers and 2nd layer printed perpendicular to the IDE fingers and an EIS of Graphene electrodes printed in parallel to the IDE fingers.

FIG. 11 depicts results from testing of sensors with negative and positive control samples. IDTE buffer was used for functionalization of the sensors.

FIG. 12 depicts results from testing of sensors with negative and positive control samples. PBS was used for functionalization of the sensors.

FIG. 13 depicts results from testing of sensors functionalized with 0.5 µM ligand DNA by dip coating, with negative and positive control samples. Positive control samples give false negative response due to insufficient loading of DNA by dip coating method.

FIG. 14 depicts results from testing of sensors functionalized with 2.5 µM ligand DNA by dip coating, with negative and positive control samples, respectively. Out of four, three positive control samples give false negative response due to insufficient loading of DNA by dip coating method.

FIG. 15 depicts DNA loading on the sensor after printing of DNA was measured by recording the change in resistance before and after printing of various layers of DNA.

FIG. 16 depicts fluorescence images of a pristine graphene sensor without DNA.

FIG. 17 depicts fluorescence images of a graphene sensor after functionalization with ligand DNA.

FIG. 18 depicts PCR amplified 72 bp ligand dsDNA.

FIG. 19 depicts PCR amplified 944 bp ligand dsDNA.

FIG. 20 depicts PCR amplified 466 bp ligand dsDNA.

FIG. 21 depicts a contact angle measurement of 10 µL water droplet with various sensor groups before and after plasma treatment used to increase the surface hydrophilicity for printing DNA.

FIG. 22 depicts a schematic illustration of the process for etching and delamination of Ti₃C₂Tₓ MXenes, and surface functionalization of Ti₃C₂Tₓ with ssDNA probes, forming ssDNA/ Ti₃C₂Tₓ biosensors for the selective detection of SARS-CoV-2 nucleocapsid (N) gene.

FIGS. 23A-F depict SEM images of (A) Ti₃AlC₂ powder and (B) accordion-like Ti₃C₂Tₓ Mxene. (C) TEM image of an exfoliated Ti₃C₂Tₓ nanosheet. (D) High-resolution TEM image of Ti₃C₂Tₓ nanosheets with a lattice distance of 0.98 nm. (E) HAADF-STEM image and corresponding elemental mapping of Ti, C, and O for the Ti₃C₂Tₓ. (F) XRD patterns of Ti₃AlC₂ and Ti₃C₂Tₓ powders.

FIGS. 24A-C depict a chemical composition and bonding configurations of as-prepared DNA-functionalized Ti₃C₂Tₓ Mxene. High-resolution XPS spectra of (A) Ti 2p, (B) C 1s, (C) P 2p, and (D) N 1s from ssDNA/ Ti₃C₂Tₓ films.

FIG. 25 depicts schematics illustrating the operation of ssDNA/ Ti₃C₂Tₓ sensors for the detection of SARS-CoV-2 nucleocapsid (N) gene.

FIG. 26A depicts a detection of SARS-CoV-2 N gene using ssDNA/Ti3C2Tx sensors showing current-voltage (IV) curves of pristine Ti₃C₂Tₓ and ssDNA-functionalized Ti₃C₂Tₓ devices.

FIG. 26B depicts a detection of SARS-CoV-2 N gene using ssDNA/Ti3C2Tx sensors showing real-time responses of Ti₃C₂Tₓ and ssDNA/Ti3C2Tx sensors toward different concentrations of SARS-CoV-2 N gene in buffer.

FIG. 26C depicts a detection of SARS-CoV-2 N gene using ssDNA/Ti3C2Tx sensors showing response as a function of SARS-CoV-2 N gene concentrations for ssDNA/ Ti₃C₂Tₓ sensors.

FIG. 26D depicts a detection of SARS-CoV-2 N gene using ssDNA/Ti3C2Tx sensors showing selectivity test of ssDNA/ Ti₃C₂Tₓ sensors toward different N gene from SARS-CoV, target SARS-CoV-2, and MERS-CoV, showing the selective detection of ssDNA/ Ti₃C₂Tₓ with SARS-CoV-2 N gene.

FIG. 26E depicts a detection of SARS-CoV-2 N gene using ssDNA/Ti3C2Tx sensors showing real-time response of ssDNA/ Ti₃C₂Tₓ sensors toward different concentrations of SARS-CoV-2 N gene in artificial saliva.

FIG. 26F depicts a detection of SARS-CoV-2 N gene using ssDNA/Ti3C2Tx sensors showing corresponding response versus concentration plot for ssDNA/Ti₃C₂Tₓ sensors.

FIGS. 27A-C depicts a detection of SARS-CoV-2 N gene from severe acute respiratory syndrome-related coronavirus 2 (SARS-CoV-2) strain 2019-n-CoV/USA-WA1/2020 showing schematic illustration of ssDNA/Ti₃C₂Tₓ biosensor for SARS-CoV-2 N gene detection

FIG. 27B depicts a detection of SARS-CoV-2 N gene from severe acute respiratory syndrome-related coronavirus 2 (SARS-CoV-2) strain 2019-n-CoV/USA-WA1/2020 showing real-time response of N gene and

FIG. 27C depicts a detection of SARS-CoV-2 N gene from severe acute respiratory syndrome-related coronavirus 2 (SARS-CoV-2) strain 2019-n-CoV/USA-WA1/2020 showing corresponding response versus concentration plot for ssDNA/Ti₃C₂Tₓ sensors.

FIG. 28 depicts a verification of the SARS-CoV-N probes for the detection of SARS-CoV-2 N gene by agarose gel electrophoresis.

FIG. 29 depicts an agarose gel electrophoresis of N gene from severe acute respiratory syndrome-related coronavirus 2 (SARS-CoV-2) strain 2019-n-CoV/USA-WA1/2020.

### DETAILED DESCRIPTION

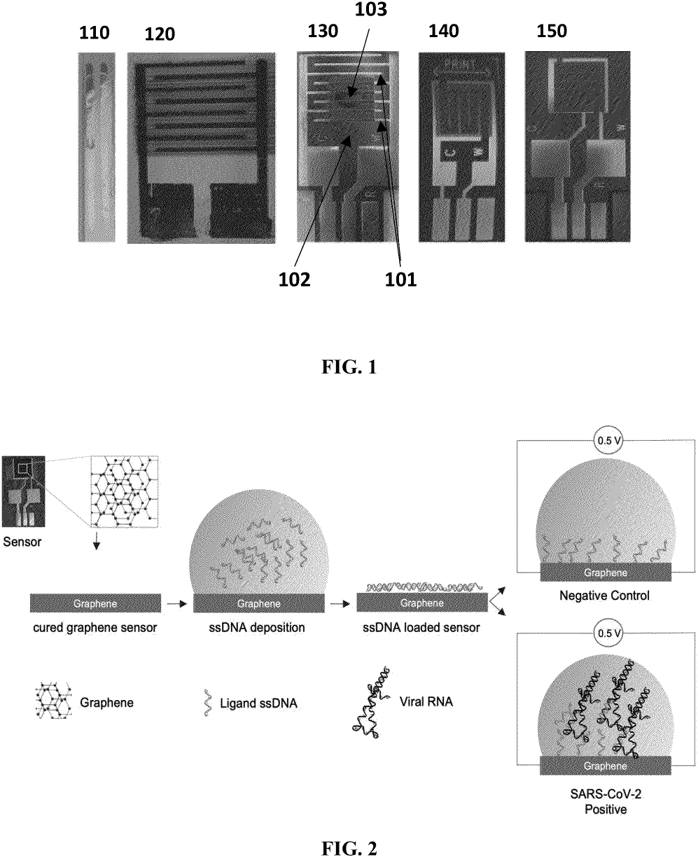

A biosensing device and method for detecting the SARS-CoV-2 virus sequence in a biological fluid are provided. In one embodiment, the device has an interdigitated electrode; a supporting flexible membrane; and an electrode material that is disposed on the interdigitated electrode and supporting flexible membrane. The device also has a sensor that reads an electrical resistance change across the interdigitated electrode after a target RNA sequence is applied to the electrode material. FIG. 1 depicts an illustrative biosensing device according to one or more embodiments provided herein. Each biosensing device can be Gen 1 110, Gen 2 120, Gen 3 130, Gen 4 140, or Gen 5 150. Each biosensing device includes, but is not limited to, an interdigitated electrode 101, a supporting flexible membrane 102, and an electrode material 103.

The sensing mechanism is based on the principle of binding single-stranded (ss) ligand DNA with the viral RNA directly released by enveloped viruses after heating at a specific temperature and time. The specific time/temperature combination for both sensor manufacturing and testing are key outcomes and parameters of the project.

In one or more embodiments, the sensor utilizes flexible electronics with graphene inks 103 printed on flexible polymeric substrates and cured, as well as electrode geometries. The 2D layered structure, electrode coating geometry, and the specific curing conditions together, enable functionalization with ssDNA primers that are binding to the graphene layers, and coming together in the construction of DNA sensor for the direct detection of enveloped RNA viruses. This printed graphene flexible electrode-DNA primer device can be used with biosensing systems for the highly specific detection of this and other enveloped RNA viruses’ nucleic acids without PCR amplification. It is important to repeat that all the fine details of the ssDNA ligand-graphene electrodes design, including curing at very specific temperatures and durations are key for the construction of DNA biosensors that are portable and can detect enveloped RNA viruses at the point of care within minutes.

The interdigitated electrode 101 can be or can include any suitable metal or other material containing one or more metals, including metal alloys. In a preferred embodiment, the interdigitated electrode 101 can be constructed from gold, silver, platinum, copper, or any combination of these metals.

The supporting flexible membrane 102 can be or can include any other polymeric film that can withstand heating in the 300-400° C. range without significant degradation of its properties. In a preferred embodiment, the supporting flexible membrane 102 can be a Kapton (polyimide film developed by Dupont) membrane.

The graphene 103 can be printed using any inkjet electronic printer or inkjet printers for materials and devices, as is known in the art.

The printable graphene ink 103 can be disposed via inkjet printing on the interdigitated electrode 101 at ambient temperatures, followed by drying at 60° C. and an essential curing step a temperature of at least 300° C. but lower than 400° C. for at least 13 hours.

In one or more embodiments, the method for detecting the SARS-CoV-2 virus sequence in a biological fluid can include disposing a biological fluid sample within the sensing device described herein.

After the biological fluid sample is located or otherwise disposed within the sensing device, the fluid sample allowed to sit or rest for a predetermined residence time. Suitable residence times are 500 sec to 1000 sec, more preferably 500 seconds.

An electrical signal, such as electrical resistance change, can be transmitted from the sensing device using any known and commercially available potentiostat.

### Example 1

The parameters of the working electrode manufacturing include three major components (1) temperature resistant polyimide substrate 101; (2) interdigitated gold electrode 102 (3) inkjet printable graphene 103. Kapton substrate with interdigitated electrodes (101) can be obtained from PCBway, China. After cleaning of the IDEs with ethanol using a moistened lint free wipe, the graphene layer was inkjet printed. The graphene ink for inkjet printing was commercially purchased. The ink contains ethyl cellulose in cyclohexanone and terpineol, inkjet printable. The product identification can be found on Sigma Aldrich, Cat No 793663. Dimatix inkjet printer was used for printing of the graphene. Based on the dimension of the IDEs obtained 6 sq. mm graphene was printed on the interdigitated fingers of the IDEs. (FIG. 1). After printing of the graphene, the IDE sheets were dried in an oven set at 60° C. for 30 min.

Table 1. Graphene inkjet printing parameters

| | |
|---|---|
| Inkjet printer: | Dimatix DMP 2850 |
| Printhead: | DMC-11610, 10 pL |
| Print resolution: | 847 dpi |
| Number of layers: | 3 |
| Time between layers: | minutes, no drying between |
| Platen temperature: | 40ºC |
| Ink cartridge temp: | 31ºC |
| Thickness: | 600 microns, |
| Jets: | 3 |
| Print origin: | X = -250 µm, Y = 400 µm |

The curing of the graphene 103 was performed at 300° C. for 13 h in ambient atmosphere. This temperature/time profile was identified as ideal for obtaining optimal DNA primer loading and is critical for sensor performance. Graphene-based flexible electrodes are cured at 300° C. for 13 h for the purpose of burning off the ink fillers, while protecting the polymeric substrate. Notably, if using whole printed sheets of electrodes, rather than individually cut electrodes, there is a very large variation in sensor performance noted. One hypothesis is that having excess polymeric substrate available during electrode curing may lead to impurities being deposited on the working sensing area, which then leads to false results and lack of reproducibility.

Different generations of sensors were built and tested with the aim of discovering the exact manufacturing parameters that would translate into a workable biosensor for SARS-CoV-2 RNA and that will also be suitable for industrial production. Generation (Gen) 1 sensor were made of CVD graphene on PET substrate with e-beam deposited gold electrodes. Gen 2 120 sensors are made of inkjet print gold IDEs on Kapton (Dupont) with inkjet print graphene layer. Gen 3 130 sensors are flex PCB from PCB Way on a generic polyimide film (flex-circuit) 0.13 mm thick with 5 sq. mm graphene layer. Gen 4 140 sensors have IDE fingers rotated in 90° compared to Gen 3 130. Gen 5 150 sensors are miniaturized version of Gen 3 130. Here it is to be noted that Gen 3 130 electrodes, individually cut, offered consistent and repeatable detection of the SARS-CoV-2 virus over multiple trials, as it will be further described in the report.

Regardless of electrode generation or manufacturing method, the prepared electrodes were cleaned with nuclease free water (NFW) before deposition of the ligand ssDNA. The ligand ssDNA is specifically binding to the SARS-CoV2 RNA and can be commercially purchased. The deposition can be followed by drying of the sensors in an oven at 60° C. for 30 min. The resistance of the sensing graphene layer between the counter and working electrode is measured and recorded. This will be considered as the baseline resistance of the sensors. 100 µL NFW can be used to clean the sensors, 3 times at least (dispense the water on WE and allow it there for 2 min). Compressed nitrogen gas was used to remove the water from the sensor surface. The resistance of the electrode can also be measured before and after sensor functionalization.

The sensors were dried at 60° C. for 30 min in an oven followed by drying under vacuum to ensure complete removal of liquid. The resistance of the graphene layer between the counter and working electrode can be measured and recorded.

The stock solution of DNA (10 µM) was prepared in IDTE buffer, pH 8.0. The DNA ligand stock solution was thawed on ice and desired concentration of the DNA ligand was prepared. IDTE buffer was used for preparing all the dilution. The prepared DNA ligand solution were heated on a heating block at 95° C. for 3 min to melt the DNA ligand. After melting the DNA ligand solution was placed on ice to snap cool for 5 min. 50 µL of the prepared ligand DNA was drop casted on the sensor and immediately dried in an oven at 60° C. With loading of the ligand DNA on the sensor the resistance of the sensor increases. The increase in resistance can be recorded.

The method for detecting SARS-CoV-2 genomic RNA included placing a small (10 µL) volume of saliva on a biosensing device, wherein said biosensing device can be pre-loaded with specific nucleic acid ligand probe. Upon introduction of sample, the ligand nucleic acid probe can be diffused freely from the sensor surface to bind with the target RNA, wherein the biosensor device can be configured to measure changes in current flow to an applied voltage.

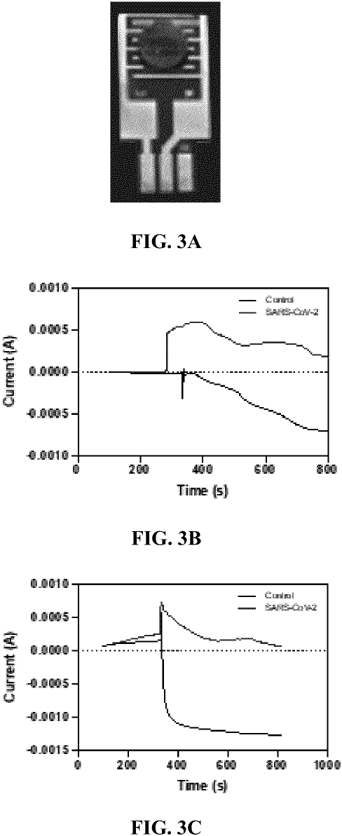

The developed sensing platform utilizes specific ssDNA probes immobilized onto the graphene (deposited on to the inkjet-printed interdigitated gold electrodes) to detect SARS-CoV-2. Graphene can be used for the sensor development, as a 2D material with higher surface area. The increased surface area provides more ssDNA probes to be immobilized on the graphene to achieve a highly sensitive sensor platform. To build a sensor system and measure the sensor response, inkjet printed inter-digitated gold electrodes were produced at the Roll-to-Roll lab. Thereafter graphene was inkjet printed on the electrodes sensing area, followed by functionalization of the surface with the probe ssDNA strands. The design of the Sensor can be described in detail in FIG. 2. The sensors employ ssDNA as the ligand probe. The ssDNA ligand has been designed to bind to the SARS-CoV-2 genomic RNA. The resistance of the pristine graphene sensor surface increases due to functionalization with ligand ssDNA. During sample testing, due to introduction of the viral genomic RNA, ligand ssDNA lifts of the sensor surface resulting in change in the sensor resistance. During sample testing a small potential can be applied and change in current due to ssDNA ligand lift off can be recorded as sensor response. A 10 µL sample drop was placed on the sensing area to test the sensor (FIG. 3A), and the sensor response was recorded. The sensor response for spiked model buffer can be depicted in FIG. 3, where the sensor response in model clinical matrix for negative control (artificial saliva) (FIG. 3B) and positive control (SARS-CoV-2 spiked artificial saliva) (FIG. 3C) samples are depicted.

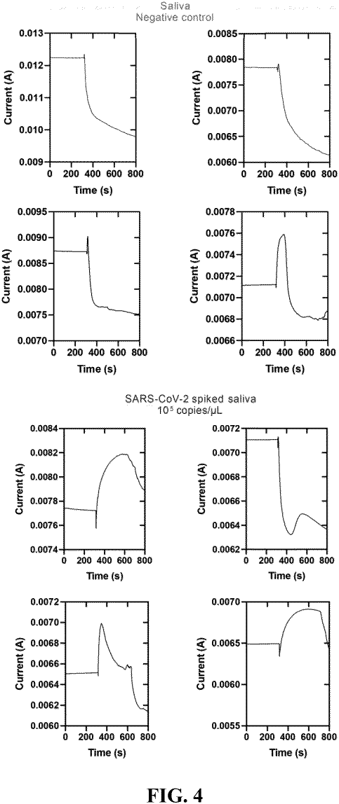

In order to develop a highly specific assay with low false negative and false positive results negative and positive control samples were tested using a quadruplex sensor system including four individual sensors, respectively. The sensor response for all four sensors were recorded simultaneously using MultiPalmSens4 from PalmSens BV. To interpret the results, sensor response from all four sensor were analyzed and best of four sensor response determines if the test result can be true negative or positive, respectively. A decrease in current response can be reported as true negative, whereas increase in current response can be reported as true positive. FIG. 4 depicts the sensor response of a quadruplex SARS-CoV-2 sensor array for the negative and positive control samples, respectively. It can be recommended that the device needs to maintain a quadruplex system to establish true positive and true negative results as 3 out of 4 repeatable results.

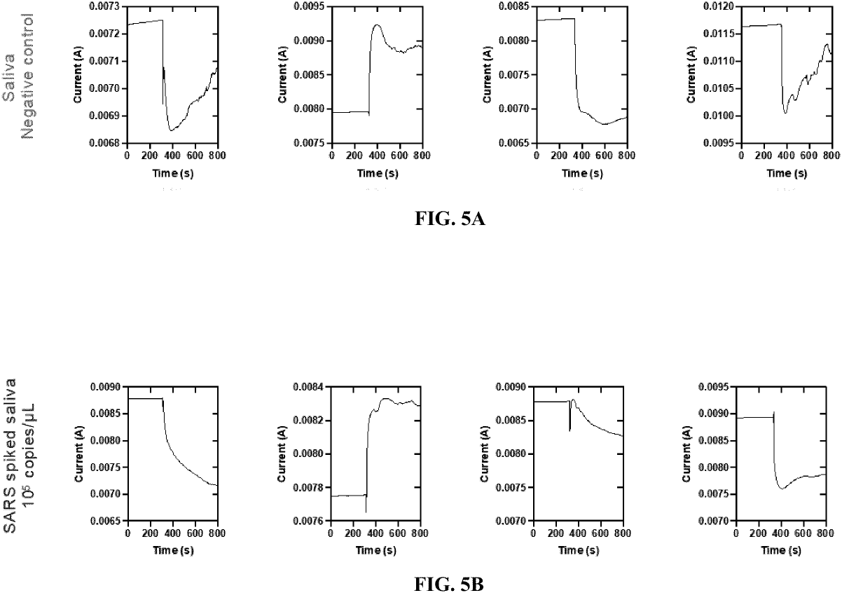

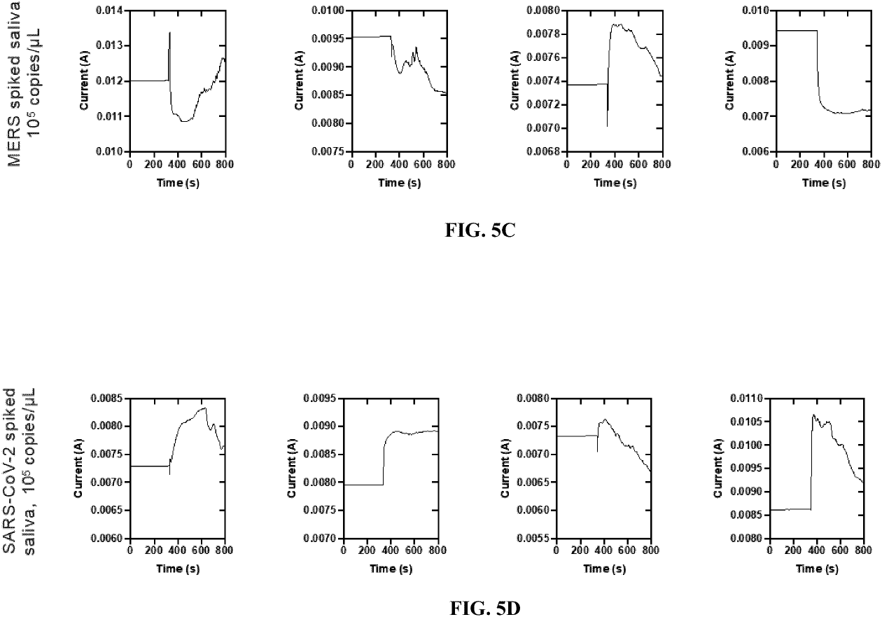

The cross reactivity of the sensor was tested with SARS-CoV and MARS-CoV virus spiked artificial saliva samples. Briefly, SARS-CoV and MARS-CoV RNA was procured from ATCC. The artificial saliva sample was spiked with the above-mentioned viral RNA to a final concentration of 10⁵ copies/µL. Ten microliters of the prepared spiked saliva sample was used as the test sample and the sensor response was recorded. Quadruplex sensor array as mentioned above, was used to determine the cross reactivity of the sensors. The sensors show a no cross-reactivity with the SARS-CoV and MARS-CoV spiked samples when analyzed as a quadruplex sensor array (FIGS. 5A-D).

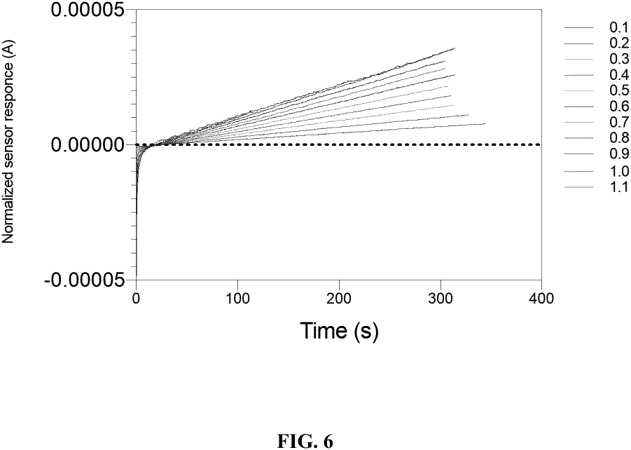

The applied positive bias for the developed sensor was causing the sensor to heat up. Therefore, a range of positive biases (0.1-1.1 V) were applied to the sensor. As shown in FIG. 6, at 0.5 V there was significant less noise and the current flow through the sensor can be about 10±4 mA depending on the baseline resistance of the tested sensor.

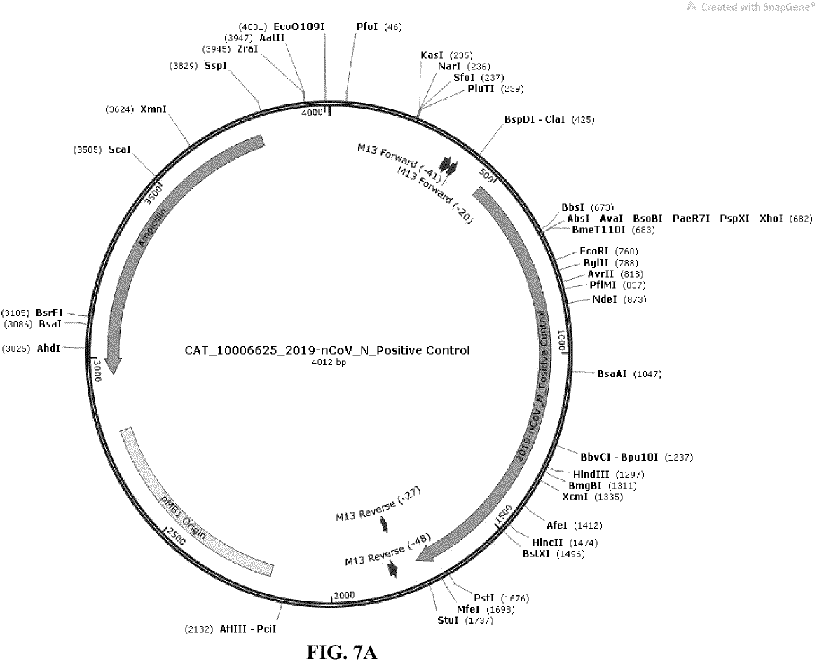

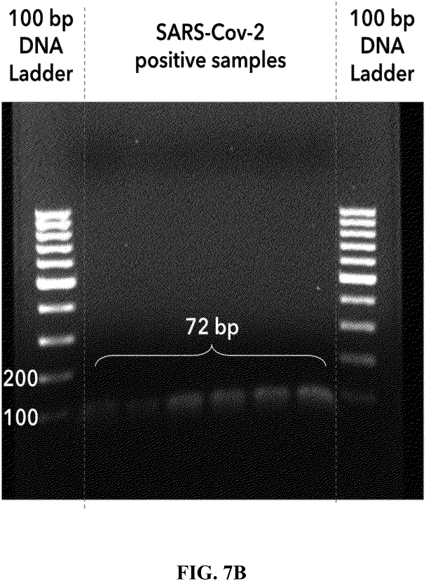

Preparation of 72 bp long DNA using PCR. SARS-CoV-2 specific 72 nucleotide long PCR product was prepared by PCR amplification. The parameters used for the PCR amplification are listed in Tables 2 and 3. Forward primer sequence: 5′-GACCC CAAAA TCAGC GAAAT-3′. Reverse primer sequence: 5′-TCTGG TTACT GCCAG TTGAA TCTG-3′. FIG. 7 depicts the DNA plasmid used as template and the amplified 72 base PCR product. After optimization of the PCR reaction, several PCR amplification of the 72 bp SARS-CoV-2 N1 gene was performed. The obtained PCR products were purified with gel extraction before being used as ligand on the sensor.

Table 2. PCR Reaction for Amplification of DNA

| Component | Concentration | Volume (µL) |
|---|---|---|
| PCR buffer | 10x | 5 |
| Forward primer | 10 µM | 1 |
| Reverse primer | 10 µM | 1 |
| dNTP mix | 25 mM of each | 0.4 |
| DNA template | 200000copies/ µL | 1 |
| Taq Polymerase | 5 U/µL | 0.5 |

Volume made 50 µL with nuclease free water

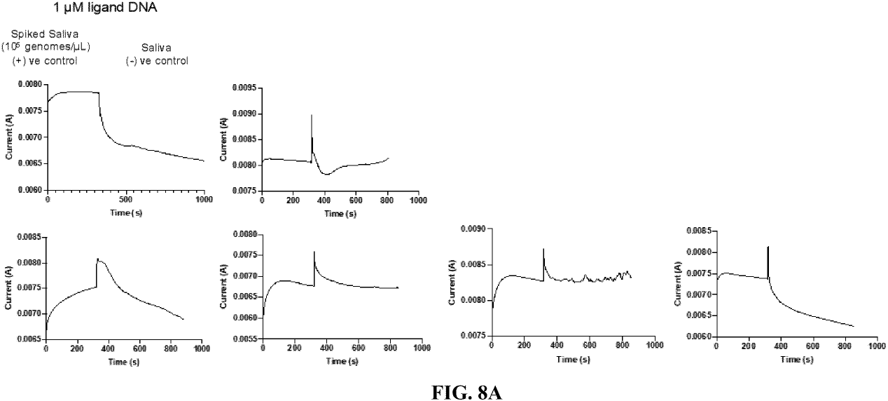

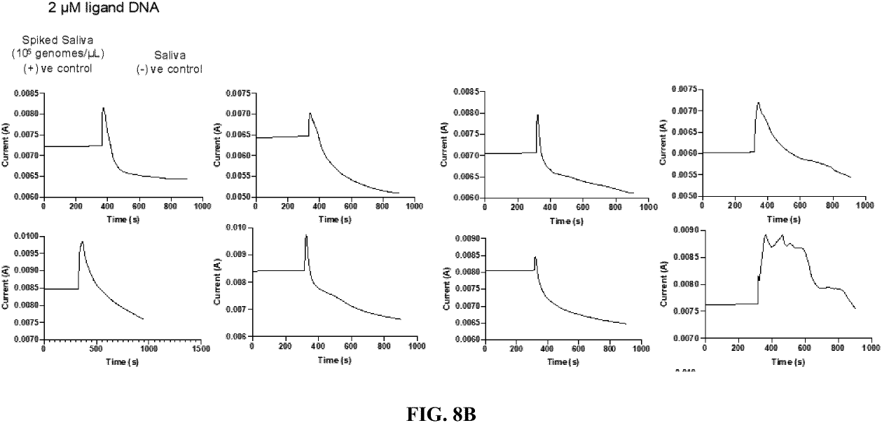

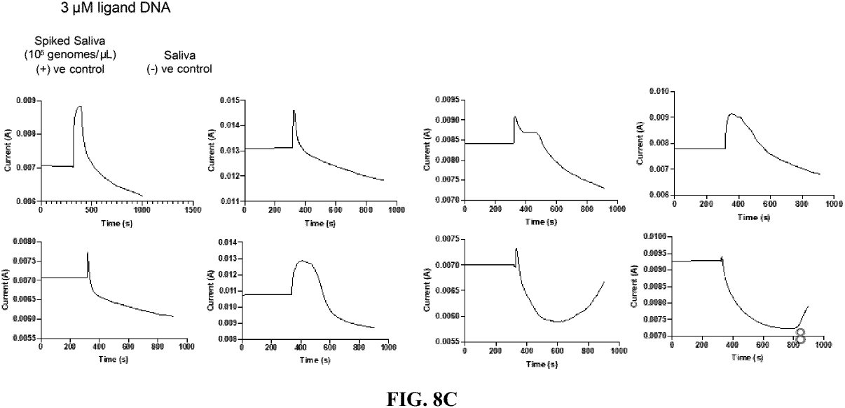

The prepared ligand DNA was used to functionalize the electrodes. Various (1, 2, 3 µM) concentrations of the ligand DNA were tested (FIG. 8). A concentration of 1 µM ssDNA ligand deposited on the electrode showed optimal performance. More specifically, this primer concentration led to the highest true negative and true positive results compared to 2 and 3 µM primer concentration, respectively.

Table 3. PCR amplification conditions

| | | | |
|---|---|---|---|
| Program Name | Temp. | Time | |
| Initial denaturation | 98° C. | 30 sec | |
| Denaturation | 98° C. | 10 sec | 35x |
| Annealing | 60° C. | 15 sec | |
| Extension | 72° C. | 30 sec | |
| Final Extension | 72° C. | 10 min | |
| Cooling | 4° C. | Cont. | |

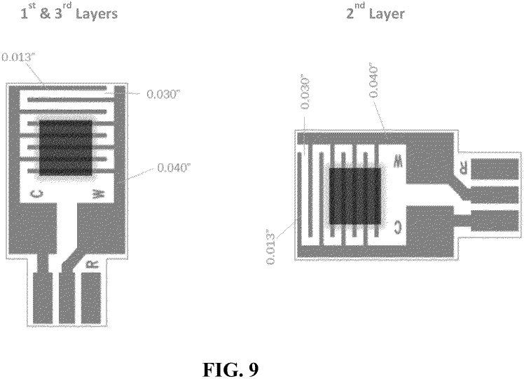

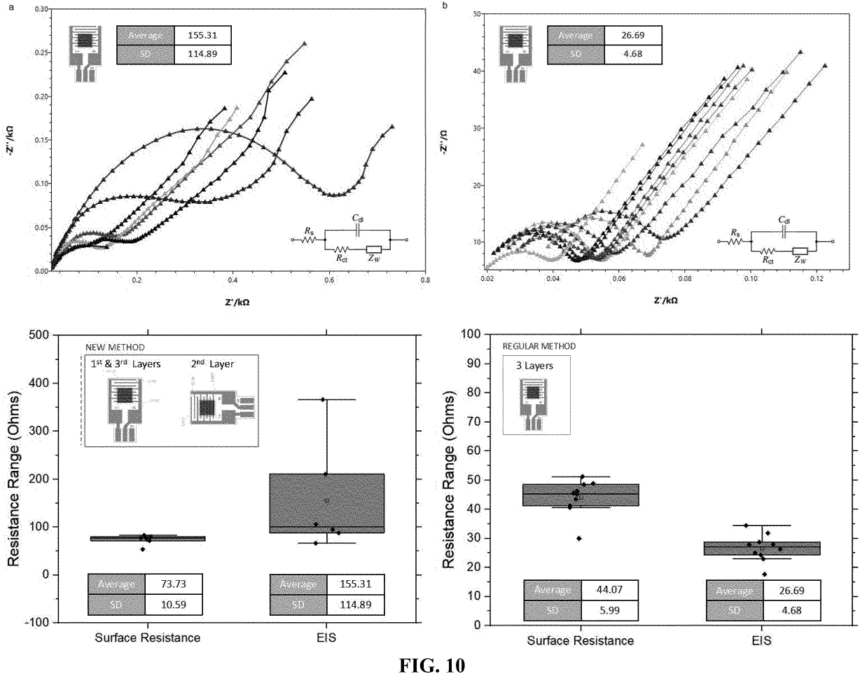

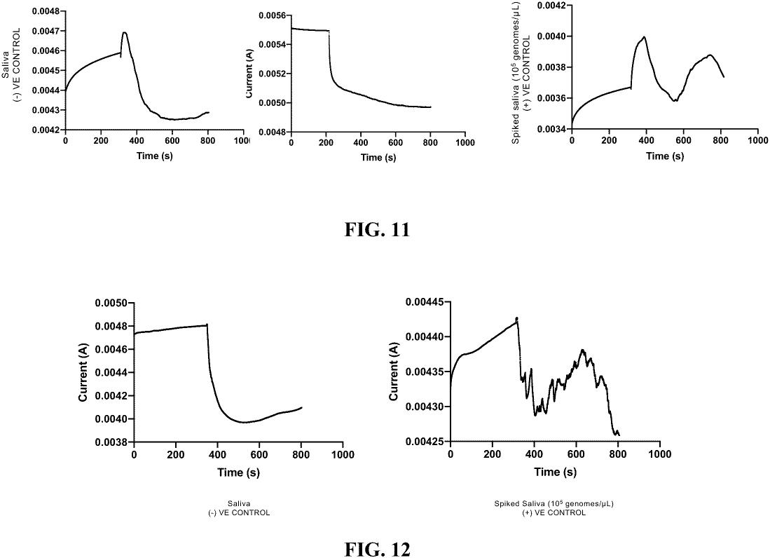

To determine the effect of print direction, Gen 3 130 (FIG. 1) sensors were produced with a) 3 layers of graphene, in parallel to the IDE fingers and b) 1st and 3rd layer of graphene in parallel to the IDE fingers and 2nd layer perpendicular to the IDE fingers (FIG. 9). By changing the angulation of the printing for each layer (scenario b) above), the electrode resistance increased, as well as the standard deviation of resistance measurements. After measuring the surface resistance and charge transfer resistance, as shown in FIG. 10, the unidirectional printing method showed the lowest average resistance and standard deviation for both measurements. Testing of these group of sensors after functionalization with ligand DNA shows higher noise and baseline resistance (FIG. 11). Functionalization of the same groups of sensors with PBS as the functionalization buffer shows increase in noise for the positive control sample (FIG. 12).

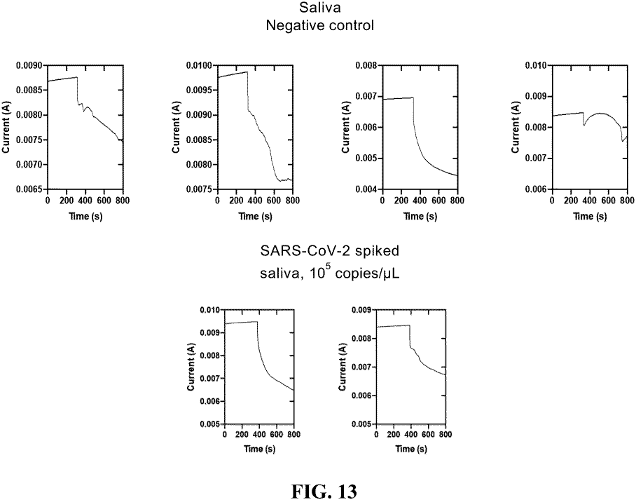

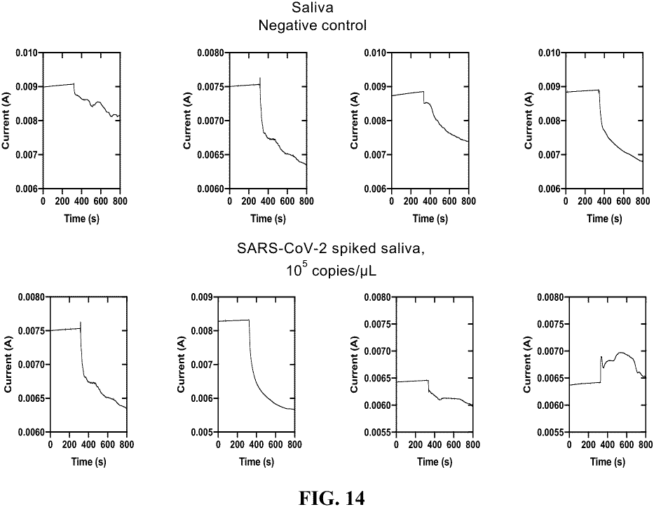

The graphene electrodes were dip coated using ssDNA primer solution prepared in IDTE buffer. Testing of this group of sensors gave false negative response. This can be due to the insufficient ligand DNA loading on the sensor surface by dip coating method. Tables 4 and 5 show the measured electrode resistance after primer functionalization via drop casting (Table 4) and dip coating (Table 5). Sensors functionalized using drop casting of ssDNA ligand on the sensor show a higher increase in resistance after primer functionalization, which indicates that this method provides a higher yield than the dip coating method. The loading of DNA was measured by recording the change in resistance before and after loading of DNA on the sensor. FIG. 13 shows that the dip coating method for primer functionalization led to false negative results, which resulted from the low yield of the functionalization reaction, which did not offer sufficient ligand DNA for the hybridization reaction with the target (viral) RNA. Increasing the primer concentration from 0.5 µM (FIG. 13) to 2.5 µM (FIG. 14) led to one positive result, which due to the 5X increase in ligand DNA concentration. This can be not ideal, nevertheless, and thus the dip coating method should not be used for future translation to large scale manufacturing.

Table 4. Resistance measurements for drop casted ligand DNA.

| Drop Casting | Before (Ω) | After 0.5 µM ligand (Ω) | Before (Ω) | After 0.7 µM ligand (Ω) |
|---|---|---|---|---|
| Minimum | 53.80 | 112.0 | 35.90 | 69.80 |
| Maximum | 82.80 | 143.2 | 84.50 | 158.0 |
| Mean | 64.48 | 121.0 | 60.84 | 112.5 |
| SD | 9.588 | 10.47 | 14.72 | 26.90 |

Table 5. Resistance values after DNA primer loading with dip coating process

| Dip coating | Before (Ω) | After 0.5 µM ligand (Ω) | After 2.5 µM ligand (Ω) |
|---|---|---|---|
| Minimum | 41.70 | 51.40 | 56.40 |
| Maximum | 63.50 | 72.60 | 79.10 |
| Mean | 48.04 | 59.82 | 64.04 |
| SD | 6.294 | 7.233 | 8.210 |

In order to provide information on various parameters to be considered to mass manufacturing, the possibility of using inkjet printing of the DNA primers was tested. This method has not been extensively reported in literature but holds promise for improving the repeatability of the primer functionalization procedure. The optimized parameters for inkjet printing of DNA are found in Table 6.

Table 6. Parameters of Inkjet Printing of DNA

| Temperature (plate and cartridge): | 25-27° C. |
|---|---|
| Filtration of the DNA solution: | sterile 0.22 µm filter |
| Voltage: | ~20 mV |
| Frequency: | 5 Hz |
| Resolution: | 847 dpi |
| Drop size: | 30 µm |
| DNA concentration: | 10 µM |
| No. of Layers: | 10 |

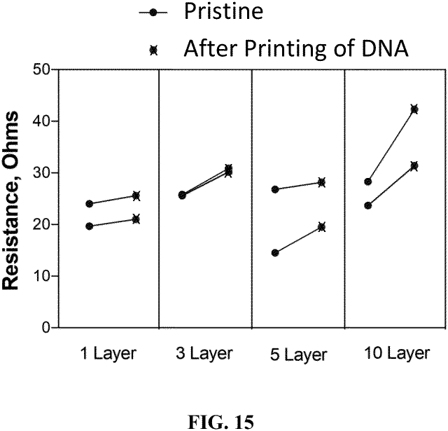

After the inkjet printing of the DNA primers, the resistance of the electrodes was measured in an effort to estimate the level of primer loading on the electrode surface. FIG. 15 shows the increase of the electrode resistance upon different DNA inkjet printing parameters. Printing five layers of DNA primers gave the highest DNA loading and can be recommended to serve as a starting point for future development.

The graphene sensor was imaged using 32 different excitation wavelengths. Graphene sensors have highest intrinsic/background fluorescence at 472 and 544 and 635 nm wavelength (FIG. 16). Alexa fluor 488 may be a suitable fluorophore for the detection of fluorescently labeled DNA on graphene surface. Another suitable fluorophore may be FITC. DNA loaded graphene sensors were also imaged at 32 different excitation wavelengths (FIG. 17). It was observed that functionalization of sensor with DNA ligand didn’t cause any interference during the imaging.

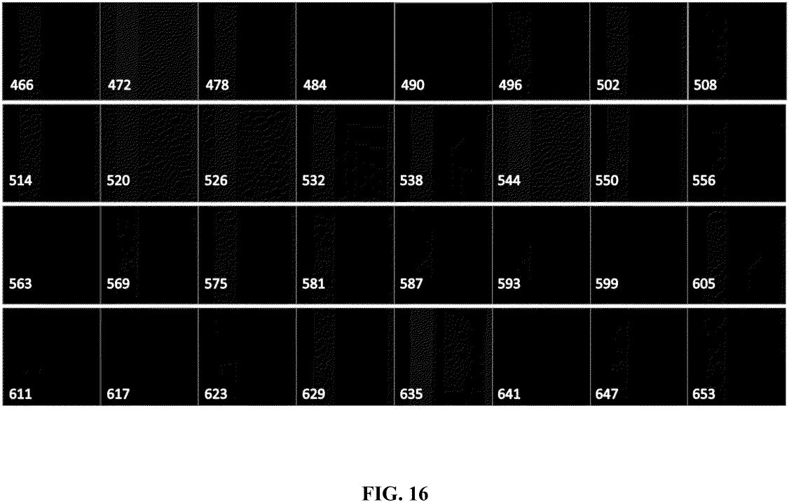

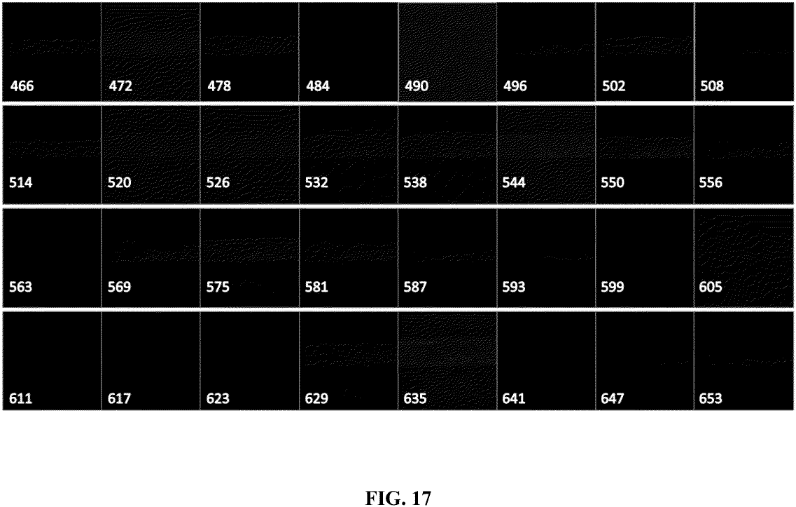

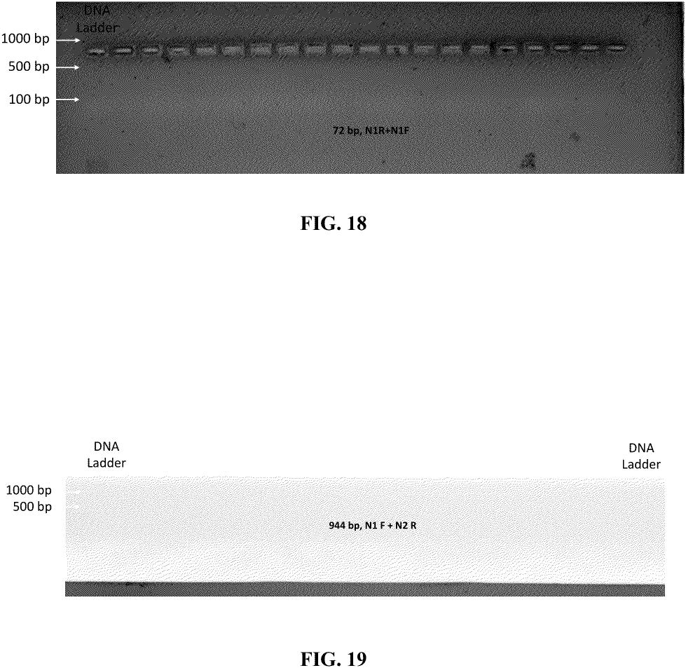

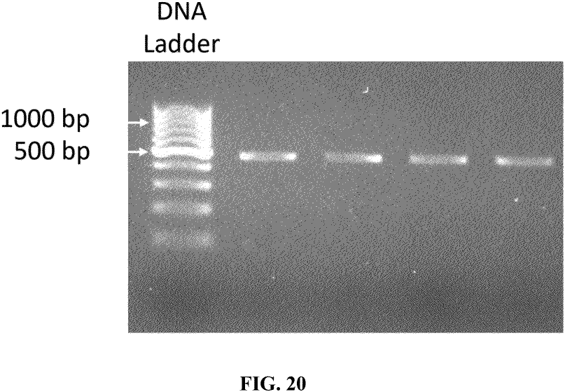

PCR amplification of the SARS-CoV-2 N gene was performed to produce various dsDNA ligand. The produced ligands are of 72 bp, 466 bp, and 944 bp long. The optimized annealing temperature for the 72 (FIG. 18), 944 (FIG. 19), and 466 (FIG. 20) base products can be 60° C., 58° C., and 58° C., respectively. The ssDNA ligand sequence used for the sensor can be 5′-TCTGG TTACT GCCAG TTGAA TCTGA GGGTC CACCA AACGT AATGC GGGGT GCATT TCGCT GATTT TGGGG TC-3′.

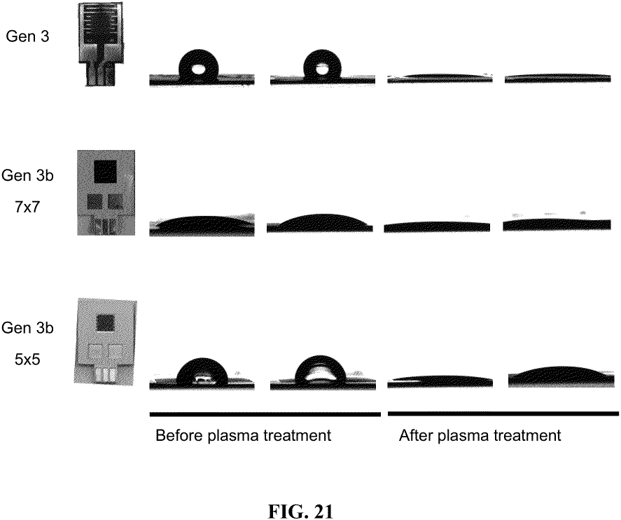

The effect of plasma treatment on the surface hydrophobicity of the graphene sensors was determined in this experiment. It was observed that after plasma treatment of the graphene sensors makes it more hydrophilic. Additionally, it was observed that Gen 3 130 sensors’ surface can be more hydrophilic than the Gen 3 130 sensors. FIG. 21 depicts the 10 µL water droplet on the sensor surface before and after plasma treatment.

### Example 2

In one or more embodiments, this disclosure describes non-obvious viral nucleic acid biosensing configurations that include two-dimensional (2D) transition-metal layered carbides and nitrides (“MXenes”) with a general formula of Mn⁺¹XₙTₓ, where M is an early transition metal, A is an A group element, X is carbon and/or nitrogen, and Tₓ represents surface functional groups such as —O, —OH and/or —F and that are functionalized with DNA primers for specificity for the viral target. The new 2D layered structure specifically enables functionalization with DNA primers that are binding in between the layers of material, thus enabling the construction of viral nucleic acid electrochemical biosensors for the detection of pathogens. The novel MXene-DNA primer sensing material 103 can be incorporated into biosensing systems for detection of pathogens, such as viruses and bacteria in saliva and nasal swabs at the lowest detection limit reported to date.

The DNA primer-MXene coating 103 presents with a higher surface area than other electrode materials, afforded by the layered structure, and can be functionalized on, which is necessary and indispensable for the construction of DNA biosensors, such as those that are able to detect infectious diseases induced by viruses and bacteria, with electrochemical characteristics enabling field deployability and point-of-care diagnostics by customizing the electrode resistance to a level that is low enough (in the range 1-8 kOhm) to be read by portable resistance readers. The electrical properties of the new MXene-DNA nanofilm 103 deposited on metallic electrodes supported by flexible substrate allows coupling with viral or bacterial DNA specific for the detection of infectious diseases, as specified above.

The DNA-functionalized Ti₃C₂Tₓ biosensor was successfully developed for a highly sensitive, selective, and rapid detection of N gene from SARS-CoV-2. The sensor response of ssDNA/Ti₃C₂Tₓ increases with the concentration of the added target DNA. A clear differentiable response in N gene of SARS-CoV-2 with the ssDNA/Ti₃C₂Tₓ sensors can be performed at a concentration as low as 100 copies/µL, indicating ultrahigh selectivity of ssDNA/Ti₃C₂Tₓ sensors for detecting SARS-CoV-2 N gene. The ssDNA/Ti₃C₂Tₓ also demonstrates a sound performance on detecting SARS-CoV-2 N gene in saliva samples. The detection mechanism of N gene from SARS-CoV-2 for DNA-functionalized Ti₃C₂Tₓ is proposed as being dominated by the hybridization of highly selective DNA probes on Ti₃C₂Tₓ surfaces with SARS-CoV-2 N gene. Under the current COVID-19 outbreak, this study shows the feasibility of developing real-time and highly reliable diagnosis device for clinical tests based on DNA-functionalized Ti₃C₂Tₓ MXenes.

The electrode material 103 can be or can include any suitable DNA functionalized two-dimensional material from the MXene family of materials. In one particular example, the DNA functionalized MXene can include Mₙ₊₁XₙTₓ, where M is an early transition metal, X is carbon and/or nitrogen, and Tₓ represents surface functional groups such as —O, —OH and/or —F and that are functionalized with DNA primers for specificity for the viral target. In a preferrable example, the DNA functionalized MXene can include Mₙ₊₁XₙTₓ, where M is Ti, X is carbon, and Tₓ represents surface functional groups such as —O, —OH and/or —F and that are functionalized with DNA primers for specificity for the viral target.

In one or more embodiments, the method for detecting the SARS-CoV-2 virus sequence in a biological fluid can include disposing a biological fluid sample within the sensing device described herein.

After the biological fluid sample is located or otherwise disposed within the sensing device, the fluid sample allowed to sit or rest for a predetermined residence time. Suitable residence times are 400 sec to 1000 sec, more preferably 500 seconds.

An electrical signal, such as electrical resistance change, can be transmitted from the sensing device using any known and commercially available potentiostat.

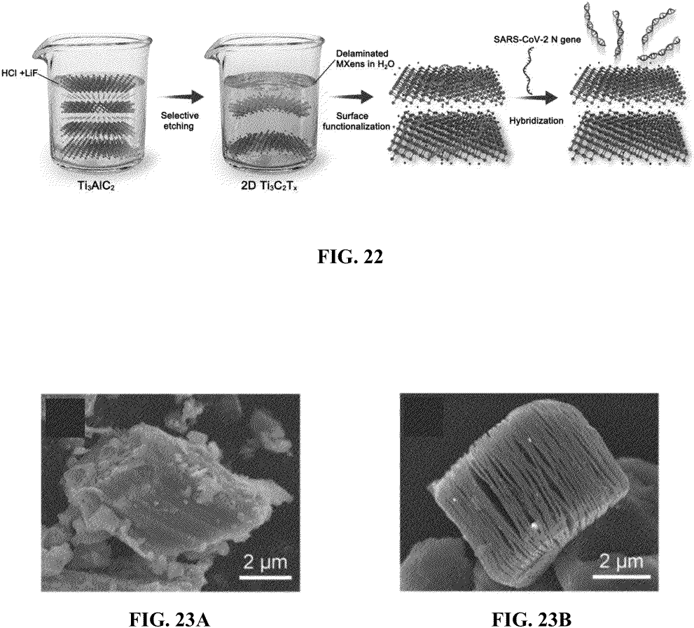

FIG. 22 shows the preparation process of ssDNA/ Ti₃C₂Tₓ biosensors for SARS-CoV-2 detection. First, the Ti₃C₂Tₓ MXene was synthesized by selective etching of Al layers from their Ti₃AlC₂ MAX phase in a HCl—LiF premixed solution and further delaminated in water by ultrasonication. The Ti₃C₂Tₓ dispersions were then spray-coated onto the gold interdigitated electrodes 101. The detailed procedures for device fabrication were given in the Experimental Section. For the selective detection of SARS-CoV-2 N gene, the Ti₃C₂Tₓ films were functionalized with probe DNAs through a noncovalent binding based on weak interactions involving the phosphate backbone and/or the nucleobases. The detection mechanism of SARS-CoV-2 N gene with ssDNA/Ti₃C₂Tₓ biosensors can be based on the sequence-specific hybridization: when the ssDNA probe was hybridized with the target SARS-CoV-2 N gene (complementary sequences), the interaction between the formed dsDNA and layered Ti₃C₂Tₓ was weakened, resulting in a desorption of the hybridized compounds off the MXene surface and thus increasing channel conductance.

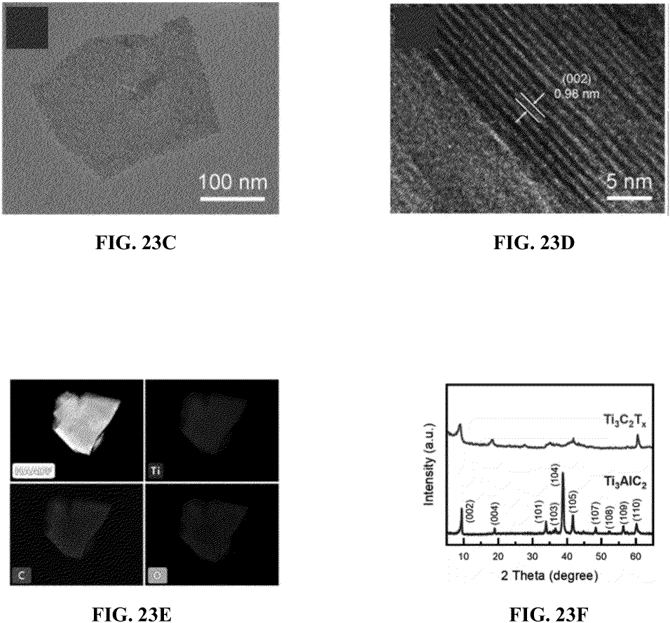

FIGS. 23A and B show the scanning electron microscopy (SEM) images of Ti₃AlC₂ and as-etched Ti₃C₂Tₓ, respectively, indicating a successful transition from a bulk Ti₃AlC₂ to an accordion-like Ti₃C₂Tₓ MXene with interlayer spaces that can serve as molecular sieving channels for hosting organic molecules and ions. Transmission electron microscopy (TEM) image of delaminated Ti₃C₂Tₓ nanosheets reveals quite thin, electron-transparent flake with a typical size of about 200 nm (FIG. 23C). The High-resolution TEM image in FIG. 23D reveals high crystallinity of the Ti₃C₂Tₓ nanosheets with a lattice distance of 0.98 nm for the (002) plane of Ti₃C₂Tₓ, consistent with XRD analysis presented later. Moreover, the high-angle annular dark-field scanning transmission electron microscopy (HAADF-STEM) imaging, along with the associated energy-dispersive X-ray elemental mapping, presented in FIG. 23E, shows a uniform distribution of Ti, C, and O elements in the Ti₃C₂Tₓ nanoflakes. X-ray diffraction (XRD) patterns of the Ti₃AlC₂ and as-etched Ti₃C₂Tₓ are displayed in FIG. 23F, further confirming the formation of Ti₃C₂Tₓ MXene. After selective etching of Ti₃AlC₂, an intense peak at 39.1°, corresponding to the (104) plane of Ti₃AlC₂, is greatly weakened, accompanied by a downshift of the (002) peak from 9.5° to 8.9°, indicating the removal of Al layers and a successful formation of Ti₃C₂Tₓ MXene.

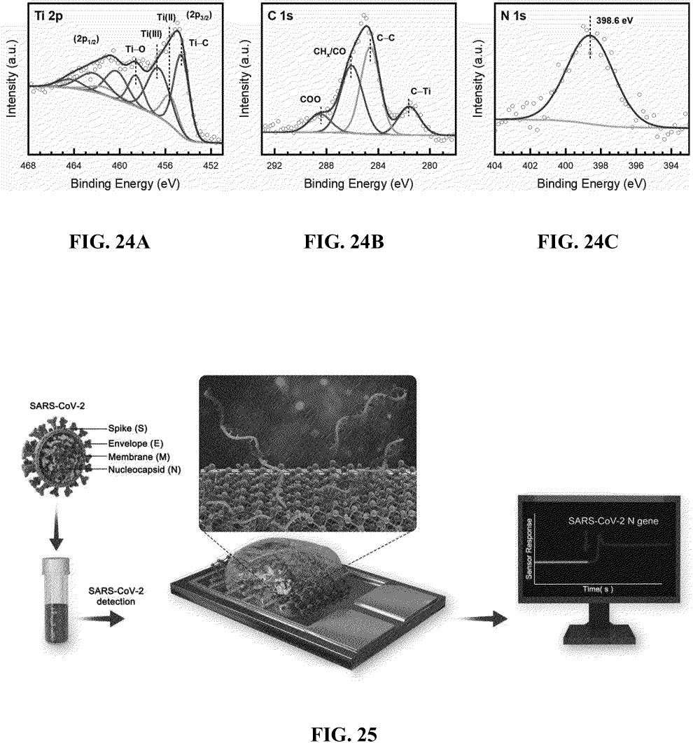

The element composition and bonding configuration of ssDNA functionalized Ti₃C₂Tₓ was further investigated by X-ray photoelectron spectroscopy (XPS). Evidenced by the high-resolution XPS results, the signals of Ti 2p, C 1 s, and N 1 s from ssDNA/ Ti₃C₂Tₓ film suggests the presence of DNA probes on Ti₃C₂Tₓ MXene after surface functionalization (FIG. 24). The high-resolution Ti 2p spectrum in FIG. 24A was fitted with four doublets (Ti 2p3/2 and Ti 2p½) with an area ratio equal to 2:1 a doublet separation of 5.7 eV. The Ti 2p spectrum was deconvoluted into four peaks center at 454.6, 455.6, 456.6, and 458.6, which corresponds to Ti—C, Ti2+, Ti3+, and Ti—O, respectively. The C 1 s spectrum in FIG. 24B was fitted with four peaks centered at 281.6, 284.6, 286.1, and 288.5 eV, corresponding to C—Ti, C—C, CHx/CO, and COO, respectively. The N 1 s peak can be a reliable indication that DNA is adsorbed on the surface of Ti₃C₂Tₓ, as nitrogen is exclusively found in the nitrogen-containing base pairs of DNA. The N 1 s spectrum in FIG. 24C can be identified with the binding energy value of 398.6 eV, clearly demonstrating the successful adsorption of DNA onto Ti₃C₂Tₓ MXene surface.

FIG. 25 shows a schematic diagram of detecting SARS-CoV-2 N gene using Ti₃C₂Tₓ -based biosensing device functionalized with probe DNAs. The desorption of ssDNA probes out of the MXene surface induced by the hybridization with complementary SARS-CoV-2 N gene results in an increase in channel current. SARS-CoV-2 genomes encode four structural proteins, including spike (S), envelope (E), membrane (M), and nucleocapsid (N). Among them, a virus surface spike protein mediates SARS-CoV-2 entry into human cells, and thus it is considered as a major target for both vaccine design and rapid diagnosis. However, the SARS-CoV-2 S gene shares 76% similarity of amino acid sequence to those of the SARS-CoV S gene. Furthermore, non-synonymous mutations changes in S protein when the SARS-CoV-2 pandemic progress. These reasons lead to complications with vaccine response and rapid diagnosis. In contrast, SARS-CoV-2 N gene is genetically stable and less nucleotide changes over time, thereby providing as a better target candidate for rapid diagnosis.

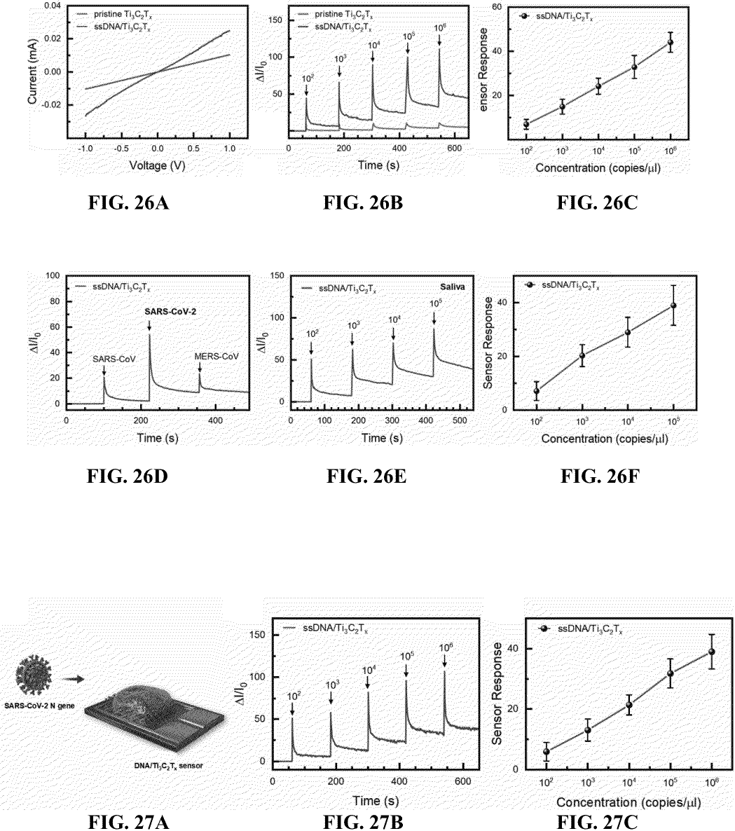

In accordance with US Centers for Disease Control and Prevention (CDC) guidelines, two specific sequences from SARS-CoV-2 were selected here: 2019-nCoV_N-F and 2019-nCoV_N-R. Recognition probes were integrated with 2D MXenes for the development of the COVID-19 sensors. To validate the presence of the ssDNA probes on the Ti₃C₂Tₓ surface, the current-voltage (IV) curves of the fabricated Ti₃C₂Tₓ before and after probe DNA functionalization were recorded. As shown in FIG. 26A, the ssDNA functionalization results in an increase in electrical resistance of the hosting Ti₃C₂Tₓ, indicating the successful introduction of ssDNA probes. To evaluate the performance of the ssDNA/ Ti₃C₂Tₓ sensors for the detection of SARS-CoV-2 N gene, the real-time measurements of the ssDNA/ Ti₃C₂Tₓ sensors were performed in a buffer solution with target SARS-CoV-2 N gene, using pristine Ti₃C₂Tₓ sensors as a control (FIG. 26B). The ssDNA/ Ti₃C₂Tₓ sensors exhibit remarkable increases in currents upon adsorption of target DNA (SARS-CoV-2 N gene) over the concentration range from 102 to 106 copies/µ; by contrast, the Ti₃C₂Tₓ sensors without probe functionalization does not show any significant response to target DNA. These findings indicate that only the ssDNA functionalized Ti₃C₂Tₓ can be sensitive to SARS-CoV-2 N gene. The responses of the ssDNA/ Ti₃C₂Tₓ sensors with concentration variations of SARS-CoV-2 N gene are shown in FIG. 26C, showing a nearly linear response to SARS-CoV-2 N gene over a wide range of concentrations. Notably, ssDNA functionalized Ti₃C₂Tₓ not only exhibits a high sensitivity to the target SARS-CoV-2 N gene, but also provides an excellent detection limit well below 100 copies/µL, which further demonstrated the feasibility of using ssDNA/ Ti₃C₂Tₓ chemiresistive sensors for quantitative analysis of viral nucleic acid.

The selectivity of ssDNA/ Ti₃C₂Tₓ sensors were validated by prepared and tested the oligonucleotide sequences from closely related N genes of SARS-CoV-2, SARS-CoV and MERS-CoV, all having a concentration of 100 copies/µL. As demonstrated in FIG. 26D, the ssDNA/ Ti₃C₂Tₓ sensor shows almost no response to SARS-CoV and MERS-CoV N genes, confirming that this platform has a sequence-specific recognition for SARS-CoV-2 N gene. The selectivity results also suggest that the SARS-CoV-2 N probes are highly specific for SARS-CoV-2 N gene, which is consistent with agarose gel electrophoresis experiments demonstrating the specificity of the SARS-CoV-2 N probes for detecting N gene of SARS-CoV-2. Moreover, the latest diagnosis of COVID-19 using a saliva sample eliminates the need of nasopharyngeal swabs, which would make the patient uncomfortable and is prone to shortage. The saliva sample has a more complex composition that could affect the sensor performance. Therefore, it is worth evaluating the dynamic response of the ssDNA/ Ti₃C₂Tₓ sensors to SARS-CoV-2 N gene in saliva. The real-time sensing results presented in FIGS. 26E and F indicate that the ssDNA/Ti₃C₂Tₓ sensors maintain a good sensitivity even with saliva sample down to the concentration of 100 copies/µL. This study provides the feasibility of detecting SARS-CoV-2 without additional PCR amplification using 2D MXene functionalized with ssDNA recognition probes as a promising pathway for rapid detection of SARS-CoV-2 gene.

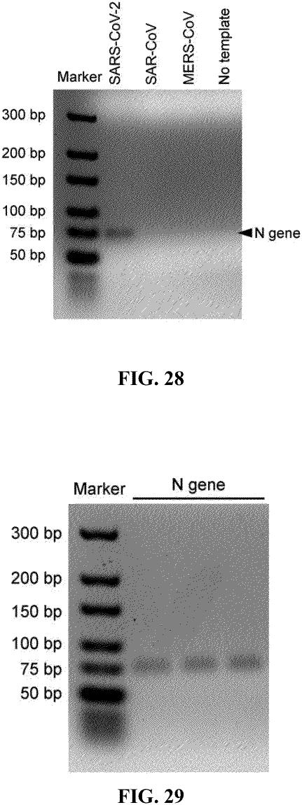

The detection of clinically relevant materials is essential and important for practical diagnostic development. Thus, the detection of N gene from severe acute respiratory syndrome-related coronavirus 2 (SARS-CoV-2) strain 2019-n-CoV/USA-WA1/2020 using ssDNA/ Ti₃C₂Tₓ sensors was performed (FIG. 27A). As SARS-CoV-2 isolation and propagation requires a Biosafety Level 3 (BSL-3) laboratory, the non-infectious SARS-CoV-2 was prepared by heating the virus to 65° C. for 30 min and thus was safe to use under BSL-1 conditions. The N gene target was confirmed by one-step qRT-PCR and agarose gel electrophoresis from severe acute respiratory syndrome-related coronavirus 2 (SARS-CoV-2) strain 2019-n-CoV/USA-WA1/2020 (FIG. 28). The SARS-CoV-2 N gene sequence contains a single band with length of 72 base pair, consistent with previous PCR results (FIG. 29). The real-time sensing of the ssDNA/ Ti₃C₂Tₓ sensor under various concentrations of N gene from severe acute respiratory syndrome-related coronavirus 2 (SARS-CoV-2) strain 2019-n-CoV/USA-WA1/2020 is shown in FIG. 27B. The sensor exhibits an evident response increased with increasing the concentration of target gene. FIG. 27C shows the relationship between the response values and target concentrations. The ssDNA/ Ti₃C₂Tₓ sensor is ultrasensitive to SARS-CoV-2 N gene with (i) a low detection limit of less than 100 copies/µL and (ii) a nearly linear response over the range of concentrations from 10² to 10⁶ copies/µL, which is suitable for detecting vial loads from the majority of reported patient at the onset of symptoms.

Materials: The Ti₃AlC₂ (2 g, particle size < 40 µm) was purchased from Carbon-Ukraine. Conductive gold ink (UTDAu25) was purchased from UT Dots (Champaign, IL, US). The plasmid controls of the viruses contain the complete nucleocapsid gene from 2019-nCoV, SARS and MERS virus, were synthesized from Integrated DNA Technologies (IDT, Skokie, IL, US). The selected oligonucleotide probes were approved by the US Centers for Disease Control and Prevention (CDC) and given in Table 7: nCOV-N Forward Probe, 5′-GACCCCAAAATCAGCGAAAT-3′, nCOV-N Reverse Probe, 5′-TCTGGTTACTGCCAGTTGAATCTG-3′. The artificial saliva was purchased from Pickering Laboratories (Mountain View, CA, US). Heat-inactivated SARS-CoV-2 (strain: 2019-nCoV/USA-WA1/2020, ATCC® VR-1986HK™) was purchased from American Type Culture Collection (ATCC, VA, US). All other chemical reagents were of analytical reagent grade and used without further purification. Ultrapure water (> 18.3 MW) was used throughout the experiment.

Table 7. Selected sequences for SARS-CoV-2 detection

| | |
|---|---|
| Description | Sequence (5′-3′) |
| SARS-CoV-2-N primer-F | GACCCCAAAATCAGCGAAAT |
| SARS-CoV-2-N primer-R | TCTGGTTACTGCCAGTTGAATCTG |
| SARS-CoV primer-F | CAAACATTGGCCGCAAATT |
| SARS-CoV primer-R | CAATGCGTGACATTCCAAAGA |
| MERS-CoV primer-F | GGCACTGAGGACCCACGTT |
| MERS-CoV primer-R | TTGCGACATACCCATAAAAGCA |

Preparation of Ti₃C₂Tₓ MXene: Ti₃AlC₂ powder was selective etched to remove Al layer in a premixed acid solution of 9 M HCl (20 mL) and LiF (3.2 g) and stirred for 200 rpm for 24 h at room temperature. The mixture was washed through several centrifugation cycles with ultrapure water until pH value of the supernatant reached approximately 6. The resulting Ti₃C₂Tₓ sediment was collected and rewashed with ultrapure water by vacuum filtration using PVDF membrane with 0.22 µm pore size and dried in vacuum at 80° C. for 24 h. To obtain delaminated Ti₃C₂Tₓ dispersion, 100 mg of Ti₃C₂Tₓ multilayer powder was sonicated in 20 ml of ultrapure water with ultrasonic bath (Branson, CPX2800H) for 1 h. The bath temperature was controlled at 4° C. to prevent restacking of the nanosheets caused by the thermal energy released during sonication. The delaminated Ti₃C₂Tₓ nanoflakes were collected for fabricating SAR-CoV-2 biosensors.

Fabrication of ssDNA/ Ti₃C₂Tₓ Sensors: First, nanogold ink was printed by a commercial inkjet printer (Dimatix DMP-2850, Fujifilm) on a polyimide substrate containing six pairs of gold interdigitated electrodes 101 with total active electrode area of 8 mm x 8 mm. Then, the Ti₃C₂Tₓ solution (5 mg/ml) was sprayed onto the interdigitated electrodes 101 by using an airbrush (G-233, Master Airbrush) for 10 s. The spray conditions were achieved with an operating pressure of 80 psi, a 0.5 mm of nozzle size, an operating distance of 30 cm between spray nozzle and substrate, and a steady moving speed of 10 cm/s in all directions. The Ti₃C₂Tₓ films were then functionalized by casting a 20 µL drop of mixed probe DNA solution (5 × 10⁻⁶ M) and dried under vacuum.

Characterization and Real-time Sensing of SARS-CoV-2 Viral Sequences: The surface morphology and microstructure of the Ti₃C₂Tₓ MXene were examined by scanning electron microscopy (SEM; S-4800, Hitachi), transmission electron microscopy (TEM; Talos 200X, FEI), high-angle annular dark-field scanning electron microscopy (HAADF-STEM), and X-ray diffractometry (XRD; X′Pert Pro, Panalytical) operated at 45 kV and 40 mA using Cu Ka. X-ray photoelectron spectroscopy (XPS; PHI 5000 Versaprobe, ULVAC-PHI) was conducted to investigate the chemical components and bonding structures. The electrical performance was measured using a source measure unit (Keithley 2400). The sensor response can be defined using the following formula; ΔI/I0 = (I-I₀)/I₀, where I and I₀ represent the real-time current and the initial current of the sensors, respectively.

It is to be understood that the present disclosure describes several exemplary embodiments for implementing different features, structures, or functions of the invention. Exemplary embodiments of components, arrangements, and configurations are described to simplify the present disclosure; however, these exemplary embodiments are provided merely as examples and are not intended to limit the scope of the invention. Each of the appended claims defines a separate invention, which for infringement purposes is recognized as including equivalents to the various elements or limitations specified in the claims. Depending on the context, all references to the “invention” may in some cases refer to certain specific embodiments only. In other cases, it will be recognized that references to the “invention” will refer to subject matter recited in one or more, but not necessarily all, of the claims. Each of the appended claims defines a separate invention, which for infringement purposes is recognized as including equivalents to the various elements or limitations specified in the claims. Depending on the context, all references to the “invention” may in some cases refer to certain specific embodiments only. In other cases, it will be recognized that references to the “invention” will refer to subject matter recited in one or more, but not necessarily all, of the claims. Additionally, the present disclosure can repeat reference numerals and/or letters in the various embodiments and across the figures provided herein. This repetition is for the purpose of simplicity and clarity and does not in itself dictate a relationship between the various embodiments and/or configurations. Moreover, the exemplary embodiments presented can be combined in any combination of ways, i.e., any element from one exemplary embodiment can be used in any other exemplary embodiment, without departing from the scope of the disclosure.

Additionally, certain terms are used throughout the present description and claims to refer to particular components. As one skilled in the art will appreciate, various entities can refer to the same component by different names, and as such, the naming convention for the elements described herein is not intended to limit the scope of the invention, unless otherwise specifically defined herein. Further, the naming convention used herein is not intended to distinguish between components that differ in name but not function.

Furthermore, in the present discussion and in the claims, the terms “including” and “comprising” are used in an open-ended fashion, and thus should be interpreted to mean “including, but not limited to.”

The term “or” is intended to encompass both exclusive and inclusive cases, i.e., “A or B” is intended to be synonymous with “at least one of A and B,” unless otherwise expressly specified herein.

The indefinite articles “a” and “an” refer to both singular forms (i.e., “one”) and plural referents (i.e., one or more) unless the context clearly dictates otherwise. For example, embodiments using “an olefin” include embodiments where one, two, or more olefins are used, unless specified to the contrary or the context clearly indicates that only one olefin is used.

Unless otherwise indicated herein, all numerical values are “about” or “approximately” the indicated value, meaning the values take into account experimental error, machine tolerances and other variations that would be expected by a person having ordinary skill in the art. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for making the measurement. Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below.

All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted.

The foregoing has also outlined features of several embodiments so that those skilled in the art can better understand the present disclosure. Those skilled in the art should appreciate that they can readily use the present disclosure as a basis for designing or modifying other methods or devices for carrying out the same purposes and/or achieving the same advantages of the embodiments disclosed herein. Those skilled in the art should also realize that such equivalent constructions do not depart from the spirit and scope of the present disclosure, and that they can make various changes, substitutions, and alterations herein without departing from the spirit and scope of the present disclosure, and the scope thereof is determined by the claims that follow.

What is claimed is:

1. A biosensing device for detecting the SARS-CoV-2 virus sequence in a biological fluid, comprising:
an interdigitated electrode;
a supporting flexible membrane;
an electrode material that is disposed on the interdigitated electrode and supporting flexible membrane, wherein the electrode material is selected from (i) two-dimensional and layered MXenes and (2) printable graphene, wherein the electrode material is functionalized with ssDNA primers specific to the SARS-CoV-2 virus sequence to be detected; and
a sensor that reads an electrical resistance change across the interdigitated electrode after a target DNA sequence is applied to the electrode material.

2. The device of claim 1, wherein the interdigitated electrode is made from gold or silver.

3. The device of claim 1, wherein the supporting flexible membrane is a Kapton membrane.

4. The device of claim 1, wherein the DNA functionalized two-dimensional and layered MXenes are of the form Mₙ₊₁XₙTₓ, wherein M comprises an early transition metal, X comprises carbon or nitrogen, and Tₓ comprises surface functional groups that are functionalized with viral RNA or DNA.

5. The device of claim 1, wherein the DNA functionalized two-dimensional and layered MXenes are functionalized by nucleic acid sequences complementary to viral RNA.

6. The device of claim 1, wherein the printable graphene is disposed on the interdigitated electrode at ambient temperature, and then cured at a temperature of about 300° C. for about 13 hours.

7. A method for detecting the SARS-CoV-2 virus sequence in a biological fluid, comprising:
disposing a biological fluid sample within a sensing device, comprising:
an interdigitated electrode;
a supporting flexible membrane;
an electrode material that is disposed on the interdigitated electrode and supporting flexible membrane, wherein the electrode material is selected from (i) two-dimensional and layered MXenes and (2) printable graphene, wherein the electrode material is functionalized with ssDNA primers specific to the SARS-CoV-2 virus sequence to be detected; and
a sensor that reads an electrical resistance change across the interdigitated electrode after a target DNA sequence is applied to the electrode material; waiting for a residence time; and reading an electrical signal from the sensor.

8. The method of claim 7, wherein the interdigitated electrode is made from gold or silver.

9. The method of claim 7, wherein the supporting flexible membrane is a Kapton membrane.

10. The method of claim 7, wherein the DNA functionalized two-dimensional and layered MXenes are of the form Mₙ₊₁XₙTₓ, wherein M comprises an early transition metal, X comprises carbon or nitrogen, and Tₓ comprises surface functional groups that are functionalized with viral RNA or DNA.

11. The method of claim 7, wherein the DNA functionalized two-dimensional and layered MXenes are functionalized by nucleic acid sequences complementary to viral RNA.

12. The method of claim 7, wherein the printable graphene is disposed on the interdigitated electrode at ambient temperature, and then cured at a temperature of about 300° C. for about 13 hours.


SEQUENCE LISTING (1 sequence)

SEQ ID NO: 1 - DNA, 1260 bp, 2019-n-CoV/USA-WA1/2020
  source 1..1260: mol_type = genomic DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa